(19) United States
(12) Patent Application Publication
(10) Pub. No.: US 2005/0209339 A1
(43) Pub. Date: Sep. 22, 2005

(54) Methods for the treatment of sinusitis

(76) Inventors: Ercem Atillasoy, Haverford, PA (US); Steven Bradford, Scottsdale, AZ (US); Lynn Day, Basking Ridge, NJ (US); Pascal Pfister, Mulhouse (FR); Amir Tavakkol, Mountain Lakes, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/519,041

(22) PCT Filed: Jul. 1, 2003
(86) PCT No.: PCT/EP03/07002

§ 371 Date: May 23, 2005

Related U.S. Application Data
(60) Provisional application No. 60/393,169, filed on Jul. 2, 2002.

(51) Int. Cl. A61K 31/137

(52) U.S. Cl. 514/649

(57) ABSTRACT

The invention provides a method of treating chronic rhinosinusitis such as allergic fungal sinusitis comprising daily oral administration of a composition from more than 500 mg to about 800 mg, preferably about 625 mg or 725 mg terbinafine base equivalent as hydrochloride, or a molar equivalent in other acid addition salt or free base form, for a duration effective to reduce the symptoms of, or eliminate chronic rhinosinusitis; it further concerns the use of terbinafine in the manufacture of a corresponding medicament, and packs containing a plurality of corresponding compositions and medicaments.

### FIELD

The invention relates to methods involved in the treatment of sinusitis, in particular chronic rhinosinusitis.

### BACKGROUND

In medical practice, sinusitis accounts for approximately 20% of office visits. Unfortunately, sinusitis is often a very frustrating and difficult to treat condition, and medical failures often become surgical patients. Hence, there is a great need for a better understanding and more effective treatments for this disease.

Sinusitis further has a very substantial healthcare impact, as evidenced by an estimated $ 5.8 billion expenditure in 1996 in the USA (J. Allergy Clin. Immunol. 103 [1999] 408-414). Approximately 12% of U.S. citizens below the age of 45 report having symptoms of chronic sinusitis. Additionally, chronic sinusitis accounts for substantial health care expenditures in terms of office visits, antibiotic prescriptions filled, lost work days, and missed school days. There were approximately 200'000 sinus surgeries performed in the USA in 1994.

Allergic fungal sinusitis (AFS) is a subset of chronic rhinosinusitis. The concept of AFS was first proposed in 1983 (Am. J. Surg. Pathol. 7 [1983] 439-443). It is caused by an intense allergic and eosinophilic inflammatory response to a fungal species. The disease appears more frequently in areas with hot, humid weather and high ambient mold spore counts. The diagnostic criteria for AFS include the presence of a chronic rhinosinusitis usually with chronic mucosal thickening on sinus radiographs or chest-thorax (CT) scans, the presence of “allergic mucin” (defined as thick sinus secretions loaded with degranulating eosinophils) and fungal hyphae within the allergic mucin (J. Allergy Clin. Immunol. 96 [1995] 24-35; Arch. Otolaryngol. Head Neck Surg. 124 [1998] 1179-1180). Nearly all patients with AFS have nasal polyps and peripheral blood eosinophilia. A positive fungal culture of the allergic mucin helps to confirm the diagnosis but is not required. The condition is also common in Europe (Laryngoscope 113 [2003] 264-269; Laryngoscope 113 [2003] 410-414).

Terbinafine is a synthetic antifungal agent of the allylamine class and is known from e.g. EP-A-24587. It is commercially available under the trademark Lamisil^{R}. Terbinafine represents a significant advance in antifungal therapy based on its potent fungicidal action in vitro and rapid clinical efficacy in various dermatophyte infections, when given orally as well as topically. Its structure is as shown in formula I

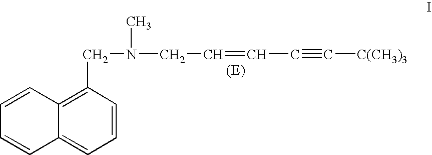

It may be in free base form or in acid addition salt form. An acid addition salt form may be prepared from the free base form in conventional manner and vice-versa. Examples of suitable acid addition salt forms are the hydrochloride, the lactate, the ascorbate and the malate, e.g. the L-(−)-hydrogenmalate. The free base and the hydrochloride and malate salts are preferred.

Terbinafine is a potent inhibitor of ergosterol biosynthesis (Ann. NY Acad. Sci. 544 [1988] 46-62), it blocks the action of squalene epoxidase, thus inhibiting the transformation of squalene to squalene epoxide. Although ergosterol synthesis is only partially inhibited, cell growth is completely arrested. This suggests that the fungicidal effect of terbinafine may be related to the accumulation of squalene, which at high concentrations may be toxic to the fungus.

The spectrum of activity of terbinafine in vitro embraces all dermatophytes of the genera Trichophyton, Epidermophyton and Microsporum. The mean minimum inhibitory concentrations for these dermatophytes range from 0.001 μg/ml to 0.01 μg/ml (Science 224 [1984] 1239-1241). Terbinafine is also active in vitro against molds and dimorphic fungi, and against many pathogenic yeasts of the genera Pityrosporum, Candida and Rhodotorula. These organisms have been implicated as associated with, or causing, chronic rhinosinusitis.

Chronic rhinosinusitis is defined as signs and/or symptoms of sinusitis persisting for more than twelve weeks.

WO 99/20261 discloses methods and materials for treating and preventing, essentially topically, non-invasive fungus-induced mucositis such as i.a. rhinosinusitis by indirect or, especially, direct mucoadministration to the paranasal cavity of antifungal agents such as i.a. terbinafine.

### SUMMARY OF THE INVENTION

It has now been found that an optimal amount of terbinafine in an oral dosage form is particularly effective in the treatment of chronic rhinosinusitis, particularly chronic rhinosinusitis that has a fungal etiology, such as allergic fungal sinusitis.

Accordingly, the invention concerns a method of treating chronic rhinosinusitis in a mammal comprising orally administering a composition comprising from more than 500 mg to about 800 mg, preferably from about 600 mg to about 800 mg, especially about 625 mg or 125 mg terbinafine base equivalent as hydrochloride per day, or a molar equivalent in other acid addition salt or free base form, for a duration effective to reduce the symptoms of, or eliminate chronic rhinosinusitis, hereinafter briefly referred to as “the method of the invention”.

The mammal preferably is human. Terbinafine most preferably is in hydrochloride acid addition salt form. The chronic rhinosinusitis preferably has a fungal etiology.

In a preferred embodiment the duration effective to reduce or eliminate chronic rhinosinusitis comprises 6 weeks and the composition is administered for a duration period of 6 weeks.

In a further embodiment, the invention is directed to a method of treating chronic rhinosinusitis in a mammal, preferably human, comprising orally administering a composition comprising from more than 500 mg to about 800 mg, preferably from about 600 mg to about 800 mg, especially about 625 mg or 725 mg terbinafine base equivalent as hydrochloride per day to said mammal for a duration period of 6 weeks.

In a further embodiment, the invention comprises the use of terbinafine in free base or acid addition salt form in the manufacture of a medicament for the treatment of chronic rhinosinusitis comprising from more than 500 mg to about 800 mg, preferably from about 600 mg to about 800 mg, especially about 625 mg or 725 mg terbinafine base equivalent as hydrochloride, or a molar equivalent in other acid addition salt or free base form.

It further concerns corresponding use in the manufacture of a medicament for the treatment of chronic rhinosinusitis comprising from more than 500 mg to about 800 mg, preferably from about 600 mg to about 800 mg, especially about 625 mg or 725 mg terbinafine base equivalent as hydrochloride, or a molar equivalent in other acid addition salt or free base form, and formulated as an oral dosage form into tablet, minitablet, powder, granule, capsule, pellet or liquid oral dosage form.

It further comprises a pack containing a plurality of terbinafine medicaments or compositions comprising from more than 500 mg to about 800 mg, preferably about 600 mg to about 800 mg, especially about 625 mg or 725 mg terbinafine base equivalent as hydrochloride, or a molar equivalent in other acid addition salt or free base form, arranged to be dispensed in a method of treating chronic rhinosinusitis in a mammal as defined above, by oral administration for a duration effective to reduce the symptoms of, or eliminate chronic rhinosinusitis, where convenient together with instructions for use, such as a calendar pack.

The term “treatment” as used herein should be understood to include prophylactic as well as curative treatment.

### DETAILED DESCRIPTION

The compositions and medicament for use in the present invention comprise terbinafine in free base or acid addition salt form and are conveniently provided in oral dosage form, e.g. formulated into tablet, minitablet, powder, granule, capsule, pellet or liquid oral dosage form, including liquid oral emulsions, preferably into tablet or minitablet form, all of which can be prepared by conventional methods. Terbinafine may be present in an amount of from about 0.1% to about 70%, e.g. from about 1% to about 60%, preferably from about 5% to about 55% base equivalent by weight based on the total weight of the composition.

The method of the invention especially is effected with the composition or medicament in tablet or minitablet form, e.g. comprising one tablet of from more than 500 mg to about 800 mg, preferably about 625 mg or 725 mg terbinafine base equivalent as hydrochloride, or a molar equivalent in other acid addition salt or free base form, or comprising two or more tablets wherein the total, combined amount of terbinafine is from more than 500 mg to about 800 mg, preferably about 625 mg or 725 mg terbinafine base equivalent as hydrochloride, or a molar equivalent in other acid addition salt or free base form.

The amount of terbinafine indicated above is indicative and may be e.g. about 610 mg, or about 710 mg terbinafine base equivalent as hydrochloride, or the molar equivalent thereof in other acid addition salt or free base form.

Administration preferably is effected once daily.

Conventional oral dosage forms may be used, e.g. marketed tablet forms.

The oral dosage forms may also be prepared e.g. as a rapidly disintegrating composition, e.g. comprising terbinafine, a disintegrant and a buffering agent, e.g. along the lines as described in WO 01/52895.

Liquid oral emulsion terbinafine compositions may also be used. The emulsion may be prepared e.g. along the lines as described in WO 01/54675. It may be either an oil-in-water or water-in-oil, preferably an oil-in-water emulsion. The emulsion may further comprise a lipophilic component, a surfactant and an emulsion-stabilizing agent, e.g. an agent preventing breakdown, e.g. creaming, coalescence or sedimentation, of the emulsion. The liquid oral emulsion compositions may further contain conventional additives such as, i.a., antioxidants, preservatives, sweetening agents, and flavoring agents.

Solid dosage forms of terbinafine for oral administration which are coated and/or multiparticulate, e.g. coated minitablets or pellets in capsules, may also be used. Such solid dosage forms may be prepared e.g. along the lines as described in WO 03/22267.

Thus, for administration of a daily dosage of 625 mg terbinafine base equivalent as hydrochloride, e.g. five conventional 125 mg tablets may be administered, each having the following composition:

| Ingredient | Amount (mg) per tablet |
|---|---|
| Terbinafine hydrochloride¹⁾ | 140.625 |
| Mg tearate (lubricant) | 2.100 |
| Hydroxypropylmethylcelluluose (binder) | 6.300 |
| Lactose monohydrate (diluent) | 21.075 |
| Sodium carboxymethyl starch (disintegrant) | 25.200 |
| Microcrystalline cellulose, granulate (diluent) | 14.700 |
| Water purified (granulating liquid)²⁾ | q.s. |

¹⁾1.125 mg terbinafine hydrochloride corresponds to 1 mg terbinafine base
²⁾removed during processing

Alternatively, for administration of e.g. a 625 mg or a 700 mg terbinafine base equivalent dosage, an appropriate number of tablets, or minitablets in a sachet or in capsules, e.g. as disclosed in Examples A, B and 1 to 16 of WO 03/22267 may be used.

Experimental Procedures for a Clinical Trial

A prospective, double-blind, parallel-group, multi-center, placebo-controlled study comparing the safety and efficacy of 625 mg of terbinafine base equivalent as hydrochloride daily, or a molar equivalent in other acid addition salt or free base form, for 6 weeks, versus placebo in the treatment of chronic rhinosinusitis is performed. The study duration is a total of 18 weeks and consists of three periods: a) screening (3 weeks, from week −3 to −1); b) treatment (6 weeks, from baseline of treatment week to week +6); and c) follow-up assessment (9 weeks, from week +7 to +15).

Sixty patients, male and female, between the age of 18 and 65 years, are randomized to receive six weeks of terbinafine therapy or placebo.

Additional inclusion criteria for admission to the study is as follows:
Patients with signs and symptoms of chronic non-invasive rhinosinusitis (CRS) for at least 3 months prior to screening. Chronic rhinosinusitis is defined by the presence of two or more major factors or one major and two minor factors as defined by the American Academy of Otolaryngology-Head and Neck Surgery (AAO-ENS); these factors are:
   1) Major factors: Facial pain/pressure*; facial congestion/fullness; nasal obstruction/blockage; nasal discharge/purulence/discolored postnasal drainage; hyposmia/anosmia; purulence in nasal cavity on examination; fever (acute rhinosinusitis only)+
      * Facial pain/pressure alone does not constitute a suggestive history for rhinosinusitis in the absence of another major nasal symptom or sign
      +Fever in acute sinusitis alone does not constitute a strongly suggestive history for acute in the absence of another major nasal symptom or sign;
   2) Minor factors: Headache; fever (all non-acute); halitosis; fatigue; dental pain; cough; ear pain/pressure/fullness
patients that have failed medical therapy and have no other therapeutic alternative than surgery;
patients with computer tomography (CT) scan evidence of sinusitis, e.g. patients with more than 25% opacification/mucoperiosteal thickening in at least two of the major paranasal sinuses;
patients with chronic sinusitis having a well documented fungal involvement, i.e. either
   with current histologic evidence of fungal hyphae obtained from the mucin of the paranasal sinuses or nasal cavity; or
   with a well-documented prior history of fungal sinusitis that is documented by histology or culture; the patients must have continuous signs and symptoms and an absence of complete clinical cure;
female patients of child bearing potential must have a negative urine pregnancy test at entry and should be practicing a medically proven form of contraception during the course of the treatment period and for 9 weeks following termination of treatment;
written informed consent must be obtained before participation in the study; a copy of each must be retained in the investigator's medical record as well as provided to the patient.

Exclusion criteria for the study is as follows:
Patients on medications found in Table 1;
patients who have had prior sinus surgery within 3 months;
women who are pregnant or breast-feeding;
patients with invasive, life-threatening fungal rhinosinusitis;
patients with current culture-proven Rhizopus fungal sinusitis;
patients with cardiac or neurological disease, or cerebral dysfunction that could, in the judgment of the investigator, jeopardize patient safety or interfere with the interpretation of the results of the study;
patients with gastrointestinal malabsorption, liver disease, nephropathy, or blood disorders that could result in the possibility of altered absorption, excess accumulation, and impairment of metabolism or excretion of terbinafine or its metabolites;
patients with hepatitis B or C;
patients with a history of abuse of alcohol, hallucinogens, or other addicting substances within the past six months;
patients with serum creatinine and/or serum glutamic oxaloacetic transaminase (SGOT, AST) and/or serum glutamic pyruvic transaminase (SGPT, ALT) values greater than the upper limit of normal (ULN) at screening;
patients who are known to be HIV positive or who are otherwise immunosuppressed;

patients with a sensitivity to terbinafine.

TABLE 1
Prohibited medications

| | Wash-out |
|---|---|
| Medication | period |
| Corticosteroid - intravenous or rectal | 1 month |
| corticosteroid - intramuscular or intra-articular | 3 months |
| high potency dermatological corticosteroid - potent or super | 1 month |
| potent by Stoughton-Cornell scale | |
| any oral antifungal | 6 weeks |
| nasal atropine, ipratropium bromide or tiotropium | 1 week |
| rifampin | 4 weeks |

Patients are seen for a total of five visits, divided into the above three periods a), b) and c):

a) Screening Period

Visit 1:A screening period (at week −3) occurs prior to the treatment period. The patient is interviewed regarding past medical history, current medical condition, and prior and current medications and vital signs. Blood samples for laboratory evaluations (including liver function tests) as well as sinus secretion collection are taken (all material must be recovered to allow for adequate specimen preparation) per the following two techniques:

1) Irrigation Technique for Sinus Secretion Collection:

1. Spray two puffs of phenylephrine hydrochloride 1% into each nostril. Wait approximately two minutes.
2. Flush each nostril with 20 ml of sterile saline using a sterile syringe with a sterile curved blunt needle (instruct the patient to take a deep inspiratory breath and hold before the injection of the saline).
3. Instruct the patient to forcefully exhale through the nose during the flushing. Collect the return (mucus) in a sterile pan.
4. Place the collected mucus into sterile centrifuge tubes for analysis.
   2) Culture Technique for Sinus Secretion Collection:

The specimen of mucus collected via the technique described above is cultured under a laminar flow hood to prevent contamination according to the following procedure:
1. Suspend the mucus specimen with an equal volume of diluted dithiothreitol (10 ml of sterile dithiothreitol with 90 ml of sterile water) and vortex for 30 seconds.
2. Allow the mixture to stand at room temperature for 15 minutes while the dithiothreitol breaks apart the disulfide bonds, in order to liquefy the mucus.
3. Centrifuge the mixture at 3000 g in a 50 ml tube for 10 minutes.
4. Discard the supernatant and vortex the sediment for 30 seconds.
5. Inoculate 0.5 ml of the prepared sediment onto different agar plates containing fungal growth medium:
   inhibitory mold agar plate containing chloramphenicol (125 μg/ml);
   inhibitory mold agar plate containing ciprofloxacin (5 μg/ml);
   brain-heart infusion agar plate containing 5% sheep blood, gentamicin (5 μg/ml), and chloramphenicol (15 μg/ml);
   brain-heart infusion agar plate containing 5% chloramphenicol (15 μg/ml), gentamicin (5 μg/ml), and cycloheximide (5 mg/ml)
   (the above antibacterial agents are utilized to prevent the growth of bacteria in the medium);
6. Incubate the plates at 30° and wait for 30 days. After the 30-day incubation period, examine at 2-day intervals and identify all cultures of fungal growth.
   b) Treatment Period

Visit 2: At visit 2 (baseline of treatment week), patients have the following procedures/evaluations performed: physical examination, vital signs, urine pregnancy test, computer tomography (CT) scan, sinus MRI T2 (optional, only in selected centers volunteering), visual acuity examinations, ophthalmoscopy and clinical evaluation of selected symptoms using visual analog scales (VAS). The patient is questioned about changes in his/her medication(s) since the screening period.

Patients who meet the inclusion/exclusion criteria are then randomized into one of two treatment groups, i.e. one group receives terbinafine treatment, and the other group receives placebo treatment.

Patients receive 625 mg terbinafine base equivalent as hydrochloride (or a molar equivalent in other acid addition salt or free base form) or placebo and take the first dose in the investigator's office. Patients are then instructed to take the 625 mg terbinafine medication (or placebo) per day, every day, for the next six weeks. The terbinafine medication can be administered as one single tablet containing the whole daily medication or, alternatively, e.g. two or more tablets, each containing less than the total daily terbinafine medication, may conveniently be combined at the time of administration such that the total dose of the multiple tablets is 625 mg terbinafine base equivalent as hydrochloride, or a molar equivalent in other acid addition salt or free base form. One such example comprises the once daily administration of five (5) 125 mg tablets of terbinafine base equivalent as hydrochloride for a duration period of 6 weeks. Administration of terbinafine in other than hydrochloride acid addition salt form is effected similarly, with the molar amounts adjusted accordingly.

Visit 3: At visit 3 (week +3 of treatment period), patients have blood samples taken for blood chemistry including liver function tests (LFT) and hematology including complete blood cell count (CBC). Concomitant medications as well as any adverse events are discussed.

Visit 4 (end of treatment; week +6 of treatment period): Patients have the following procedures/evaluations performed: physical examination, vital signs, laboratory evaluations, visual acuity examinations, ophthalmoscopy and clinical evaluation of selected symptoms using visual analog scale (VAS), sinus CT scan, sinus MRI T2 (optional, only in selected centers volunteering), nasal irrigation as well as patient and physician overall evaluation assessments.

In a subset of centers, a biopsy of the nasal mucosa is effected in order to assess the terbinafine levels in the mucus and the nasal mucosa. The technique for biopsy of the nasal mucosa in the physician's office is as follows:
1. Spray the nasal cavity with a combination of a topical anesthetic mixed with a vasoconstrictor (e.g. 1% Xylocaine with 0.001% epinephrine).
2. Wait 5 minutes and then either re-spray or place a cottonoid soaked with the same solution into the middle meatus.
3. Wait 5 minutes.
4. With appropriate visualization, a biting forceps is used to carefully remove the mucus in addition to inflamed nasal mucosa overlying the Bulla ethmoidalis. If not accessible, the nasal mucosa between the middle turbinate and the lateral nasal wall will be sampled.
5. Harvested mucosa and mucus are placed into separate vials for analysis.
   c) Follow-Up Assessment Period

At visit 5 (week +15 from baseline visit) the following procedures are performed: physical examination, vital signs, clinical evaluation of selected symptoms using VAS (as compared to baseline and end of treatment), as well as patient and physician overall evaluation assessments.

Evaluation of Results

The primary variable in determining the efficacy of terbinafine in the treatment of chronic rhinosinusitis is the change from baseline in the total opacification score of the 5 major right and left sinuses (frontal, maxillary, anterior and posterior ethmoid and sphenoid) using a score on a scale of 0 to 4.

Secondary variables in determining the efficacy of terbinafine in the treatment of chronic rhinosinusitis include:
1) the change from baseline in the total right and left obstruction score of the frontal recess, middle meatus, infundibulum, and sphenoethmoid recess, each scored as 0 or 1 (CT scan);
2) the patients overall evaluation of sinusitis;
3) the physician's overall evaluation of sinusitis;
4) the patient's evaluation of therapeutic response;
5) the physician's evaluation of therapeutic response;
6) the change from baseline in facial pain/pressure VAS;
7) the change from baseline in facial congestion VAS; and
8) the change from baseline in nasal discharge VAS.

The results of the study indicate that, relative to the primary and to the secondary variables of efficacy, a regimen comprising once daily 625 mg oral terbinafine base equivalent as hydrochloride (or a molar equivalent in other acid addition salt or free base form) for a period of 6 weeks provides a therapeutically effective regimen for the treatment of chronic rhinosinusitis.

1-3. (canceled)

4. A method for the treatment of chronic rhinosinusitis comprising administering to a patient in need thereof about 625 mg or about 725 Mg terbinafine in free or acid addition salt form.

5. A pharmaceutical composition comprising about 625 mg or about 725 mg terbinafine in free or acid addition salt form in oral dosage form, said oral dosage form selected from a tablet, minitablet, powder, granule, capsule, pellet and liquid oral dosage form.

6. A pack containing pharmaceutical compositions according to claim 2 arranged to be dispensed in a method of treating chronic rhinosinusitis in a mammal comprising orally administering a composition comprising about 625 mg or about 725 mg terbinafine base equivalent as hydrochloride per day, or a molar equivalent in other acid addition salt or free base form, for a duration effective to reduce the symptoms of, or treat chronic rhinosinusitis, where convenient together with instructions for use, such as a calendar pack.
